(19) United States
(12) Patent Application Publication
(10) Pub. No.: US 2012/0003715 A1
(43) Pub. Date: Jan. 5, 2012

(54) Process for Producing Recombinant Lyosomal Using Insect Larvae

(75) Inventors: Victor Infante Viñolo, Puerto Santa Marie (Cadiz) (ES); Rosa Maria Osuna Aguilar, Puerto Santa Marie (Cadiz) (ES); Juan Jose Infante Viñolo, Puerto Santa Marie (Cadiz) (ES)

(73) Assignee: Bioorganic Research and Services S.L., Jerez de la Frontera (ES)

(21) Appl. No.: 13/255,863

(22) PCT Filed: Mar. 13, 2009
(86) PCT No.: PCT/ES09/70063

§ 371 Date: Sep. 9, 2011

(51) Int. Cl. C12N 9/12; C12N 9/38; C12N 9/16; C12N 9/78; C12N 9/40; C12N 9/26; C12N 9/44; C12N 9/00; C12N 9/24; C12N 9/20

(52) U.S. Cl. 435/194; 435/200; 435/207; 435/196; 435/198; 435/208; 435/201; 435/210; 435/183; 435/227

(57) ABSTRACT

A method for expressing and industrial scale production of recombinant lysosomal enzymes utilizing insect larva including the steps of infecting an insect larva population of the species Spodoptera littoralis via a recombinant baculovirus liquid suspension which permits the expression of at least one gene coding for a protein of interest, at least one of these genes coding for a lysosomal enzyme expressed in the insect larva, collecting of the previously infected insect larva expressing in a sufficient significant quantity the protein of interest and recovering the protein of interest from the collected insect larva.

### BACKGROUND TO THE INVENTION

1. Field of the Invention

The present invention refers to the use of an insect larvae based baculovirus expression system as a system for the expression and industrial production of recombinant lysosomal enzymes; pharmaceutical compositions of the enzymes produced with this system and the application of the same for treatment of lysosomal storage disorders in humans and animals.

2. Related Art

The possibility to express a protein of choice in another organism provided enormous advances for biochemistry and was accompanied by the rapid development of expression vectors and techniques which permitted an increase both in the quantity of recombinant protein generated but also in the diversity of organisms used as biofactories. After laboratory development, these technology platforms developed for the production of proteins of industrial interest should be based on methods which assure controllable and safe production with high performance at the lowest possible cost and facilitate the production of the protein, all this while avoiding degradation problems.

The most commonly used expression systems for industrial scale production are in bacteria, yeast, fungi and mammalian cells cultivated either in monolayer or in suspension culture, the production in insect cell culture and more recently, the production in plants and transgenic animals.

Generally, and especially in cells of higher organisms, a correct functionality depends upon posttranslational modifications which the proteins are subjected to by various determined enzymes. These modifications may be naturally distinct, such as glycosylation, acetylation, phosphorylation, etc. Within these examples the glycosylation (addition of sugars to determined amino acids) which converts a protein into a glycoprotein is highlighted as being especially important. Glycosylation is the most common posttranslational modification, being, in the majority of cases, fundamental for biological activity of the resultant glycoprotein.

Although mammalian and insect cell systems can be used for the production of glycosylated proteins, their use requires complex platforms with associated high costs. Also, the bioreactors in which the cells are grown function during extended periods of time, running the risk of cell culture contamination.

Success in the production of functional proteins in insect cells gave rise to the search for alternatives to the expression of recombinant proteins in this system resolving the drawbacks, especially the elevated cost of production and scalability issues. The alternative solution was to use the baculovirus based expression system but to infect insect larvae, which in this case are used as non-fermentative biofactories. Insect larvae have been converted into the system of choice for the production of a large number of proteins requiring posttranslational modifications which cannot be performed in bacterial expression systems (Ailor, E. et al, 1999 Current Opinion Biotechnology, 10(2)142-5). The production of proteins which have been produced in insect cells and which require these types of posttranslational modifications for complete functionality has already been demonstrated (Jarvis, D. L. et al, 1997, Glycobiology, 7(3):433-43; O'Reilly, D. R. et al, 1994, Oxford University Press, New York). There exists numerous examples of proteins which have been expressed successfully via the baculovirus system and it has also been demonstrated that baculovirus have potential to be carriers of genes in human genetic therapies (Boyce and Bucher, 1996, Proc. Nat. Acad. Sci. USA, 93:2348-52).

The production of mammalian recombinant glycoproteins, including human proteins, demonstrating the functionality of the native protein has been widely described using the insect larvae baculovirus expression system. The use of this system to express biopharmaceuticals began with work by Maeda et al (1985) where they expressed functional human α-Interferon in silk-worm larvae (Bombyx mori (B. mori)) (Maeda, S. et al, 1985, Nature, 315:592-4). Some other examples of recombinant proteins successfully expressed in insect larvae include: Hemagglutinin from the flu virus (Kuroda et al 1989, Journal of Virology, 63:1677-85) in Heliothis virescens; human adenosine deaminase (Medin, J. A. et al, 1990, Proc. Natl. Acad. Sci., 2760-64), human activin C (Kron, R. et al, 1998, J. Virol. Methods, 72:9-14) the p30 protein from African Swine Flu (Barderas, M. G. et al, 2000, J. Virol. Methods, 89:129-36), antibody fragment against the botulinum toxin (O'Connell, K. P. et al, 2007, Mol. Biotechnol., 36:44-51) in Trichoplusia ni; Hepatitis B surface antigen (Higashihashi, N, et al, 1991, Journal of Virology Methods, 35:159-67), human beta interferon (Deng, J. et al, 1995, Clin. J. Biotechnol., 11:109-17), growth hormone (Sumathy, S. et al, 1996, Protein Expr. Punt, 7:262-68), human butyryl-cholinesterase (Wei, W. L. et al, 2000, Biochem. Pharmacol., 60:121-26), trypsin II inhibitor from Momordica charanti (Sato, S. et al, 2000, Biosc. Biotechnol. Biochem., 64:393-98), Canine VP2 parvovirus (Choi, J. Y. et al, 2000, Arch. Virol., 145:171-77), human fibroblast growth factor (Wu, X. et al, 2001, Protein Expr. Purif., 21:192-200), bovine interferon C (Murakami, K. et al, 2001, Cytokine, 13:18-24) in B. mori; phosphorylated triacylglycerol lipase (Arrese and Wells 1994) in Manduca sexta or Hepatitis E surface antigen in Spodoptera litura (Sehgal, D. et al, 2003, Protein Expr. Purif, 27:27-34).

Lysosomal diseases are genetic diseases derived from the poor functioning of the lysosomes, principally caused by a deficiency in the expression of the enzymes which they contain. Within this group of diseases the lysosomal storage diseases (LSDs) stand out, these are defined by poorly functional enzymes which gets translated into an uncontrolled accumulation of the particular enzyme substrate in the lysosome. The accumulation of these macromolecules in different tissue organs give rise to progressive physical disorders which can lead to death.

Until now there have been more than forty LSDs catalogued, which according to their prevalence are classified as rare diseases but taken together affect 1 in every 5000 births. This percentage increases considerably within the Jewish population. Those diseases with the highest prevalence and best studied are Gauchers' disease (deficiency in the glucocerobrosidase enzyme), Fabrays' disease (α-galactosidase A problem), Pompe disease (α 1-4 y 1-6 acid glucosidase), Hunters syndrome (iduronate 2-sulfatase) and Sanfilippo disease (α-N-acetylglucosaminidase) (Winchester, B. et al, 2000, Biochem. Soc. Trans., 28: 150-4).

Currently, the most common treatment for the LSDs is the use of enzyme replacement therapy (ERT) with some examples already on the market and yet others in clinical development. For example, exogenous α-L-iduronidase is used for the treatment of Hurler syndrome (Cleary, M. A. and Wraith, J. E., 1995, Acta Pediatr., 84:337-339) or the more known use of β-glucocerebrosidase for the treatment of symptoms of Type-I Gauchers' disease in the form of Ceredase® prepared from placenta or the recombinant Cerezyme® (Cox, T. M. and Schofield J. P., 1997, Baillieres Clip. Hemata, 657-89).

Until now all the recombinant lysosomal enzymes either in development or on the market are produced in mammalian cell culture. Notwithstanding, associations of those affected and government sanitary agencies are demanding the use of more efficient production systems such as that described in the present invention, in order to provide a widespread and sustainable treatment for patients.

The studies described by Davis T. R. et al, (1995) (Davis, T. R. et al, 1995, In vitro Dev. Biol., 31; 659-63) demonstrate the potential of the expression of recombinant proteins in insect larvae to generate in a natural manner a glycosylation pattern rich in complex mannose residues. In this way, cells with mannose receptors are able to import in a selective manner the proteins expressed and containing exposed mannose residues. Within the typology of cells capable to perform this task are, amongst others, endothelial cells, epithelial cells and immature dendritic cells. It is also known that the macrophages of the endothelia are systems especially affected by LSD diseases and that ERTs are directed specifically towards these systems and show high affectivity (Hahn at al, 1998, Proc. Natl. Acad. Sci., 95: 14880-85).

These data suggest that the recombinant lysosomal enzymes produced by the system described in this presented invention would present a glycosylation pattern highly suitable for use as an ERT for LSDs, conferring an important competitive advantage of the larval expression system over others which require the introduction of modifications to obtain glycosylated proteins rich in exposed mannose residues.

These considerations are also presented as advantages by the invention described in the patent U.S. Pat. No. 7,241,442 which proposes the use of insect cell cultures, especially SF9 and Sf21 cells, for the production of lysosomal enzymes. As a significant improvement, this present invention proposes for the first time the use of insect larval systems, entailing an option with a greater industrial level viability.

### SUMMARY OF THE INVENTION

The present invention proposes the application of a baculovirus expression system in insect larvae as a means to optimally produce lysosomal enzymes. The characteristics of the presented system for the production of recombinant proteins contain different phases: obtaining a suspension of recombinant baculovirus capable of expressing at least one gene coding for a protein of interest, with at least one of these genes coding for a lysosomal enzyme; the infection of insect larvae utilizing the aforementioned recombinant baculovirus suspension; following the evolution of the infection in the infected larvae to determine the optimum production of the protein of interest; the collection of the larvae and subsequent purification of the enzyme maintaining the natural glycosylation pattern generated by the expression in the insect larvae.

The invention refers to expression of lysosomal enzymes in said larval system and their industrial production. Some examples include in a nonexclusive manner the following human lysosomal enzymes: galactocerebrosidase, hexosaminidase A, hexosaminidase B, N-acetylgalactosamine-6-sulphatase, cysteine transport proteins, N-acetyl-alpha-D-glucosaminidase, NPC1 protein, alpha-1,4-glucosidase, alpha-1,6-glucosidase, acidic alpha-1,6-glucosidase, alpha-L-iduronidase, idunorate sulphatase, heparin N-sulphatase, galactose-6-sulphatase, acidic beta-galactosidase, beta-glucurinidase, N-acetylglucosamine-1-phosphotransferase, alpha-N-acetylgalactosaminidase, acidic lipase, acidic lysosomal ceramidase, acidic sphingomyelinase, glucocerebrosidase, galactosylceramidase, alpha-galactosidase A, acidic beta-galactosidase and beta-galactosidase.

Pharmaceutical compositions of lysosomal enzymes obtained via this system are included in this invention, as is also the therapeutic applications in human or animal subjects. In particular the invention concerns its application to ERT for the treatment of symptoms of LSDs.

An advantage of the present invention is to employ the insect larval expression system as a means to obtain economically viable industrial quantities of human lysosomal enzymes.

An advantage of the present invention is to employ the insect larval expression system as a means to obtain human lysosomal enzymes with the glycosylation pattern especially suitable for its application in treatment of symptoms associated with LSDs.

An advantage of the present invention is to employ the insect larval expression system as a means to obtain human lysosomal enzymes and to use them for the production of antibodies specific for use in medical diagnostic systems.

An advantage of the present invention is to employ the insect larval expression system as a means to obtain human lysosomal enzymes and apply them to any commercial use which implies the hydrolysis of substrates.

Other advantages and objects of the present invention will be apparent to those knowledgeable in the arts of the techniques after reviewing this document or by the implementation of this invention.

### DETAILED DESCRIPTION OF THE INVENTION

Traditionally, the most commonly used expression systems for the industrial production of biotechnological pharmaceuticals are bacteria, yeast, fungi and mammalian cells grown in monolayer or in suspension. Notwithstanding, in the past few years alternative technologies have appeared on the market such as the baculovirus based systems in insect cell culture or larval system and, more recently, the production in transgenic plants and animals.

With respect to the final product obtained, one of the principal differences between the distinct expression systems currently available is the posttranslational modification pattern. Within this field it is known that the baculovirus expression systems give rise to a naturally occurring glycosylation pattern rich in exposed mannose residues (Davis T. R. et al, 1995, cited above).

Due to this, the present invention describes for the first time the application of insect larval expression as an optimal process for the industrial production of recombinant lysosomal enzymes, pharmaceutical compositions of the said produced enzymes and their application to the treatment of LSDs.

According to the objectives of the present invention any methods of larval based baculovirus expression systems would be an appropriate production method, if the resulting pattern of glycosylation is not significantly different from the glycosylation pattern derived from the posttranslational modifications produced naturally by insect cell culture.

In the context of the present invention a method of baculovirus expression in the larval system is defined as a process whereby a determined protein of interest is produced in insect larvae after being infected by a recombinant baculovirus vector which has been specifically designed for said purpose, such as is described in Patterson et al, (Patterson, R. M. et al, 1995, Environmental Health Perspectives, 103:756-9).

Within the available non-limiting options, the following invention proposes an optimised method containing the following steps: the construction of a baculovirus containing information to express with high efficiency at least one enzyme of interest using an AcMNPV (Autographa californica (Multiple) Nuclear Polyhedrosis Virus) baculovirus or similar; obtain a suspension of recombinant virus in any state, being of special interest in this invention the obtention of recombinant baculovirus in the germination state via replication of the virus in insect cell cultures, especially in cultures of Sf9 or Sf21 cells from Spodoptera frugiperda or HighFive from Trichoplusia ni and the subsequent purification of said virus; infection of larvae of Lepidoptera, especially larvae of the Trichoplusia ni or Spodoptera littoralis species via parenteral injection of an effective dose of the baculovirus suspension or an aerosol treatment with the viral suspension; purification of the recombinant protein from the infected larvae via conventional means known to those familiar to the art to obtain a crude extract and subsequently purify to homogeneity. The time point to collect and process the infected larvae to maximise the quantity of protein of interest is determined individually and empirically for each recombinant baculovirus and for each Lepidoptera species used as a biofactory.

With respect to conventional techniques, for the objective of the present invention it is not considered critical the use of one method or another of baculovirus expression in larval systems other than those listed in the bibliography, although the use of suitable conditions and the described procedures in the patent application PCT/ES2009/070061 are proposed.

The present invention proposes the following non-limiting examples of expression of human lysosomal enzymes in the larval system: galactocerebrosidase, hexosaminidase A, hexosaminidase B, N-acetylgalactosamine-6-sulphatase, cysteine transport proteins, N-acetyl-alpha-D-glucosaminidase, NPC1 protein, alpha-1,4-glucosidase, alpha-1,6-glucosidase, acidic alpha-1,6-glucosidase, alpha-L-iduronidase, idunorate sulphatase, heparin N-sulphatase, galactose-6-sulphatase, acidic beta-galactosidase, beta-glucurinidase, N-acetylglucosamine-1 phosphotransferase, alpha-N-acetylgalactosaminidase, acidic lipase, acidic lysosomal ceramidase, acidic sphingomyelinase, glucocerebrosidase, galactosylceramidase, alpha-galactosidase A, acidic beta-galactosidase and beta-galactosidase.

The present invention proposes the use of the aforementioned list of enzymes in ERT for the treatment of symptoms linked to LSDs.

According to data presented in the present invention, the enzyme is catalytically active end presents a glycosylation pattern rich in mannose oligosaccharides. Bonten et al, (2004) (Bonten, E. J. et al, 2004, FASEB J. 18(9):971-3), demonstrate that the catalytically active β-galactosidase produced in insect cells is specifically captured by mice macrophages which are affected by Gauchers' disease. They demonstrate that the specific capture is due to the exposed mannose residues produced as a consequence of naturally occurring insect posttranslational modifications. Once treated with the insect produced enzyme, the mouse macrophages which present a deficiency in the β-galactosidase enzyme present an activity for this enzyme 145% that of the activity coming from healthy mice (Bonten, E. J. et al, cited above). The macrophages internalise the human recombinant β-galactosidase produced in insect larvae in a highly efficient manner. Given that larvae of S. littoralis produce β-galactosidase highly efficiently and that the therapeutic activity of this lysosomal enzyme is attributed to its activity principally in macrophages, the larval produced human β-galactosidase could be more effectively used than the same enzyme produced in other expression systems for the treatment of symptoms of LSDs via the administration of an effective therapeutic dose of the enzyme where the subject suffers from a deficiency of the enzyme β-galactosidase. In general the enzymes, parts of the enzymes or biologically active derivatives of the enzymes produced in this system would form part of a pharmaceutical composition to be administered. The pharmaceutical composition, treatment, dosage, administration form, as well as any other aspect related to the administration of the pharmaceutical composition, would be case dependent.

The following table presents as non-excluding examples, the diseases to treat with the corresponding enzymes, which produced using the system described in this presented invention, can be used as a principal active component in ERT (Table 1):

TABLE 1
Lysosomal storage diseases and the corresponding replacement enzyme

| Disease | Replacement enzyme |
|---|---|
| Krabbe | galactocerebrosidase |
| Tay-Sachs (GM2 Ganglyosidosis) | hexosaminidase A |
| Sandhoff | hexosaminidase B |
| Morquio A | N-acetylgalactosamine-6-sulphatase |
| Morquio B | beta-galactosidase |
| Cystinosis | cysteine transport proteins |
| Sanfilippo B | N-acetyl-alpha-D-glucosaminidase |
| Niemann-Plck C | protein NPC1 |
| Pompe | acidic alpha-1,6-glucosidase |
| Hurler | alpha-L-iduronidase |
| Hunter | idunorate sulphatase |
| Sanfilippo A | heparin N-sulphatase |
| Sly | beta-glucoronidasa |
| Mucolipidosis II | N-acetylglucosamine-1- |
| | phosphotransferase |
| Schindler | alpha-N-acetylgalactosaminidase |
| Wolman | acidic lipase |
| Farber | acidic lysosomal ceramidase |
| Niemann-Pick A y B | acidic sphingomyelinase |
| Gaucher | glucocerebrosidase |
| Fabry | alpha-galactosidase A |
| Goldberg | acidic beta-galactosidase |

The objective of the following example is to improve the understanding of that written in the present invention but is not limiting in character nor should be used to limit the scope of the claims of the same.

It is obvious that experts in the material could consider variations and modifications without altering the scope of the invention according to what is described.

### Example 1

The example describes the application of the method proposed in the present invention for the production of the recombinant human lysosomal enzyme β-galactosidase (EC 3.2.1.23), an enzyme which catalyses the hydrolysis of βgalactosides into monosaccharides. Amongst its substrates are ganglioside GM1, lactose, lacosylceramides and some glycoproteins (Kohji, I. et al, 1990, Biochemical and Biophysical Research Communications, 167:746-53). The partial or total deficiency of β-galactosidase is related with various lysosomal diseases, for example mutations in the β-galactosidase gene locus give rise to gangliosidosis GM1 and Morquio B (Neufeld, E. F. and Muenzer, J., 2001, The mucopolysaccharidoses. In: The metabolic bases of inherited disease. New York: McGraw Hill; 3421:52). Gangliosidosis or Goldberg syndrome is caused by an excess in the degradation of β-galactosidase due to a deficit in the lysosomal protecting protein or cathespin A (EC 3.4.16.5) in combination with a deficit in neuraminidase (EC 3.2.1.18), which is also related to a deficit in the lysosomal protecting protein or cathespin A (Okamura-Oho, Y. et al, 1994, Biochim Biophys Acta, 1225:244-54).

For the expression of the recombinant human lysosomal enzyme β-galactosidase the system described in the solicited patent PCT/ES2009/070061 is recommended.

The cDNA coding for the recombinant human lysosomal enzyme β-galactosidase was obtained from the pGEM (GP8) plasmid (Oshima, A. et al, 1988, Biochemical and Biophysical Research Communications, 157:238-44) and was cloned into the pVL1392 (BD Biosciences, San Diego, Calif., USA) baculovirus transfer vector. The vector DNA containing the corresponding DNA insert and linearised AcMNPV viral DNA were used in a co-transfection of Sf21 cells from the ovary of Spodoptera frugiperda for selection of recombinant virus into which the aforementioned insert DNA substitutes by homologous recombination at the locus for the polyhydrin of AcMNPV. The resultant AcMNPV-β-galactosidase baculovirus is amplified in Sf21 cell culture in Sf-900 II media (Invitrogen, Carlsbad, Calif., USA) as described in O'Reilly et al, (1994) (O'Reilly et al, 1994, Oxford Univ. Press, ISBN: 0195091310). The production of the recombinant enzyme is analysed via Western Blot as per methodology similar to that described by Itoh et al, (1990) (Itoh et al, 1990, Biochem. Biophys. Res. Commun.167(2):746-53), but using the polyclonal antibody raised against human β-galactosidase (Abeam, ref. ab4761). The functionality of the enzyme produced in the cell cultures was also assayed via a β-galactosidase assays which is described below. After the amplification of the recombinant baculovirus is selected for catalytically active recombinant human β-galactosidase, a suspension of this virus is obtained at a titre of 2×10¹ plaque forming units/ml, the titre being measured via the BaculoELISA Titer kit following the instructions from the provider (Clontech).

300 larvae of S. littoralis in the L5 stage obtained by conventional breeding techniques were infected by an aerosol dispersion system. 1.5 ml of the recombinant baculovirus suspension was used for the infection of groups each containing 30 individuals. The infected larvae are incubated in insectary conditions with a 14 hour of light per day photoperiod, 26° C. and a constant relative humidity of 60% in 25 ml polypropylene individual incubation cells with 10 ml of artificial diet prepared according to the method of Vargas-Osuna et al, (1988). Post infection, groups of 20 larvae are selected and frozen for the purification and activity analysis of the β-galactosidase at various time points, namely; 0, 24, 48, 72, 84, 96, 108, 120 and 144 hours post infection (hpi). Crude extracts are prepared by resuspending the larvae in DP1 Buffer (50 mM TRIS pH 7.9, 500 mM NaCl, 10 mM Imidazole and 2 mM 2-Mercaptoetanol) using a mortar, maintaining the suspension cold at all times. The homogenisation is completed by applying the use of the Ultra Turrax T-18 during 10 minutes at 20,000 revolutions per minute (rpm). The homogenate is then filtered through a 0.25 μm filter and the filtrate used as the crude extract for the quantification of the β-galactosidase.

The activity of the β-galactosidase is analysed via the liberation of o-nitrophenol (a yellow colour) in an assay solution of 1.5 mM OPNG (o-Nitrophenyl-β-D-galactopyranoside, Sigma) in 0.1M Sodium Phosphate Buffer pH6.0 in a 65° C. water bath. Different tubes containing 1 ml of the assay solution of the assay are tempered for a few minutes and the reaction is started by the addition of different dilutions of the crude extract previously obtained from the larvae; control reactions have the DP1 Buffer added instead of the crude extract. After a 10 minute incubation period the reactions are stopped by the addition of 1 ml of 1M Sodium Carbonate, the tubes are briefly centrifuged at 3,500 rpm for 5 minutes and the supernatant fraction is measured for absorbance at 405 nm in a spectrophotometer. The absorbance values at 405 nm are converted to o-nitrophenol concentrations bearing in mind the extinction coefficient for ONPG of 4376 mM⁻¹ cm⁻¹. The total protein concentration is measured via the Bio-Rad Protein Assay Kit using Bovine Serum Albumin (BSA) as standard. All enzyme activity assays were performed in triplicate and the quantity of the enzyme produced in the different crude extracts analysed via Western Blot as previously indicated.

The maximum β-galactosidase activity was registered for those larval extracts collected at 120 hpi, where the average weight of the larvae was 790 mg t 85 mg. The yield was 223 μg±45 μg of β-galactosidase/larva with a registered average activity of 240,000 nmoles of o-nitrophenol/hr/gram of larva (240,000 units/gram of larva).

Using the 120 hpi crude extracts collected from the larvae, the glycoprotein is purified off a column of Concanavalin A (ConA) (GE-Healthcare) using 1M Methyl-α-D-Mannopyranoside (Sigma). The glycoprotein solution is then dialysed against Phosphate Buffered Saline (PBS) and stored at −80° C. until its use in capture assays of the β-galactosidase by macrophages from mice affected by EAL.

The purified β-galactosidase is subjected to de-glycosylation with N-glycanase and endoglycosidase H (New England Biolabs) following methods described by Martin et al, (1988) (Martin B. M. et al, 1988, DNA, 7(2):99-106). The purified and untreated enzyme produced in larvae gave a molecular weight of approximately 82 kDa in Western Blot assays using the aforementioned antibody. Post treatment with N-glycanase to eliminate asparagine oligosaccharide residues, a band of 80 kDa was observed, whilst treatment with endoglycosidase H gave rise to bands of 80 and 81 kDa respectively. These results confirm that the recombinant β-galactosidase produced in the larvae contain chains of oligosaccharides rich in mannose residues as a result of posttranslational maturing conducted in the insect.

The β-galactosidase produced in CHO cell culture and used in ERT in Gauchers' patients, requires in vitro treatment with N-glycanase enzymes to allow the exposure of the mannose residues. In this example, the larval produced enzyme is used directly given the natural glycosylation in insects consist basically in paucimannose patterns with exposed mannose residues to be captured by the mannose receptors of the macrophages, following the strategy of Bonten et al, (2004) (Bonten, E. J. et al, 2004, FASEB J. 18(9):971-3).

Whilst in certain features of this invention have been described in detail, it would be evident to experts in the field as to the non-limiting and the clarifying effect of the cited examples. Numerous modifications and variations of the present invention would be evident in light of data shown and therefore are included with the scope of the following claims.

What is claimed is:

1. A method is defined for the expression and industrial scale production of recombinant lysosomal enzymes in insect larva comprised of the following steps:
a. The intentional infection of an insect larva population of the species Spodoptera littoralis via a recombinant baculovirus liquid suspension which permits the expression of at least one gene coding for a protein of interest, at least one of these genes coding for a lysosomal enzyme expressed in the insect larva.
b. The collection of the previously infected insect larva expressing in a sufficient significant quantity the protein of interest.
c. Recovery of the protein of interest from the collected insect larva.

2. The method according to claim 1 where the insect larva are to include but are not limited to of the Spodoptera littoralis, Plutella xylostella, Bombix mori, Idalima leonora, Periscepta polysticta, Laspeyresia pomonella, Manduca sexta, Spodoptera exigua, Lymantria dispar, Heliothis virescenses, Helicoverpa zeas or Trichoplusia ni species.

3. The method according to claim 1 where the enzyme which is subject to expression is mammalian human or animal in origin.

4. The method according to claim 1 where the enzyme which is subject to expression is includes but is not limited to: galactocerebrosidase, hexosaminidase A, hexosaminidase B, N-acetylgalactosamina-6-sulphate sulphatase, cysteine transport proteins, N-acetyl-alpha-D-glucosaminidase, Niemann-Pick, Type C1 protein (NPC1), alpha-1,4-glucosidase, alpha-1,6-glucosidase, acid alpha-1,6-glucosidase, alpha-L-iduronidase, iduronate-2-sulfatase, heparan N-sulphatase, galactose-6-sulphatase, acidic beta-galactosidase, beta-glucuronidase, N-acetylglucosamine-1-phosphotransferase, alpha-N-acetylgalactosaminidase, acidic lipase, lysosomal acid ceramidase, acidic sphingomyelinase, glucocerebrosidase, galactocirebrosidase, alpha-galactosidase, acidic beta-galactosidase and beta-galactosidase.

5. The method according to claim 1 where the lysosomal enzymes obtained form part of pharmaceutical compositions.

6. The method according to claim 1 where a part of the lysosomal enzymes obtained form part of pharmaceutical compositions

7. The method according to claim 1 where derivatives of the lysosomal enzymes obtained form part of pharmaceutical compositions

8. The method according to claim 1 where the pharmaceutical compositions referred to in claims 5, 6 and 7 are used in enzyme replacement therapies.

9. The method in claim 1 where the pharmaceutical compositions referred to in claims 5, 6 and 7 are used for the treatment of symptoms of lysosomal storage diseases.

10. The method in claim 1 where the pharmaceutical compositions referred to in claims 5, 6 and 7 are used for the treatment of symptoms of includes but is not limited to: Krabbe disease, Tay-Sachs (GM2 gangliosidosis), Sandhoff disease, Morquio Type-A disease, Morquio Type-B disease, Cystinosis, Sanfilippo Type-B syndrome, Niemann-Pick Type-C disease, Pompe disease, Hurler's disease, Hunter disease, Sanfilippo Type-A syndrome, Sly disease, Mucolipidosis Type-II disease, Schindler disease, Wolman disease, Farber disease, Niemann-Pick Type-A and B disease, Gaucher disease, Fabry disease, and Goldberg disease.
